(19) United States
(12) Patent Application Publication
(10) Pub. No.: US 2011/0224771 A1
(43) Pub. Date: Sep. 15, 2011

(54) CATHETER

(76) Inventor: Michael Schwager, Winterthur (CH)

(21) Appl. No.: 12/740,101

(22) PCT Filed: Jul. 22, 2008
(86) PCT No.: PCT/CH08/00327

§ 371 Date: Apr. 27, 2010

(30) Foreign Application Priority Data
Oct. 29, 2007 (CH) 1675/07

(51) Int. Cl. A61M 25/10; A61F 2/84; A61M 25/098; B23P 17/04

(52) U.S. Cl. 623/1.11; 604/96.01; 604/103.1; 604/103.02; 604/529; 29/428

(57) ABSTRACT

The invention relates to a catheter for introducing into a hollow human or animal organ. Said catheter comprises a catheter tip that protrudes forwards and a region that is encased by an expandable tubular hollow body and that is situated behind the catheter tip. Said catheter is characterised in that a wire spiral is located in the encased region. It can also be advantageous to provide a radiopaque ring, in particular a platinum, gold or tungsten ring, on a hollow shaft of the catheter, together with several rib-type projections running around the hollow shaft and projecting from the latter, in a region in front of and/or behind the radiopaque ring.

### TECHNICAL FIELD

The invention relates to a catheter to be inserted into a hollow organ of a human or an animal, comprising a forwardly projecting catheter tip, wherein a region encased by an expandable tubular hollow body is arranged behind the catheter tip. Additionally, the invention relates to a catheter to be inserted into a hollow organ of a human or an animal, comprising a forwardly projecting catheter tip and a hollow shaft. Furthermore, the invention relates to a method for producing a catheter.

### PRIOR ART

Catheters are basically thin tubules or tubes that are inserted into hollow organs or vessels of humans or animals for therapeutic or diagnostic purposes. More particularly, urethras, esophagi, bile ducts or blood-carrying arteries of humans and/or animals can be explored, penetrated, emptied, filled and/or rinsed using catheters. Here, the ability to insert a catheter substantially depends on the external diameter or the cross-sectional area of the catheter in the insertion direction. Additionally, good flexibility of the catheter is likewise decisive for the latter to be inserted easily; however, it should be noted that a certain amount of stiffness is by all means desirable, particularly in the region of the catheter tip, so that the catheter can also be moved through stenoses in the hollow organ or in the vessel.

Particularly catheters that additionally have expandable tubular hollow bodies, such as balloons or medical implants, are problematic during the insertion. Although such hollow bodies can be folded-up relatively tightly and can be arranged around the catheter in a space-saving fashion, this results in relatively large external diameters of the catheters due to the wall thickness of the hollow bodies and the remaining catheter elements.

However, due to the mechanical requirements with respect to the catheter shafts, the diameter thereof cannot be reduced arbitrarily in order thus to minimize the external diameter of the catheter. That is to say that if the dimensions of the catheter shafts are too small, the ability to insert them likewise suffers due to the lack of stiffness.

Therefore, there still is a need for catheters with expandable tubular hollow bodies that can be inserted in an improved fashion.

### DESCRIPTION OF THE INVENTION

It is therefore an object of the invention to develop a catheter belonging to the aforementioned technical field, which catheter can be inserted into hollow organs of humans or animals in an improved fashion.

The solution to the object is defined by the features of claim 1. In accordance with the invention, a wire helix is arranged in the encased region.

In this context, a wire helix is understood to be a hollow-cylindrical structure formed by a wire wound around a longitudinal axis in a helical fashion.

Surprisingly, it was found that the external diameter of the catheter could be reduced within the encased region by using a wire helix without the buckling stability of the catheter being adversely affected by this. Since the wire helix has high flexibility in both a longitudinal direction and transverse direction compared to a tube or tubule of the same diameter, the wire helix only adversely affects the flexibility of the catheter in an insignificant fashion. However, together with the expandable tubular hollow body, there is in any case sufficient stiffness and this allows the insertion of the catheter even through very constricted spots in a hollow organ. A wire helix is additionally advantageous in that free space is available inside, in which further components of the catheter, such as radiopaque markers or fluid-conducting shafts, can be arranged.

In order to produce the catheters according to the invention, it is merely necessary to surround a wire helix with an expandable tubular hollow body.

A longitudinal axis of the wire helix is preferably arranged coaxially to a longitudinal axis of the catheter. Such an arrangement allows an assembly that saves as much space as possible. Moreover, if the tubular hollow body is borne directly on the wire helix and fitted to the latter, the risk of the tubular hollow body slipping during the insertion of the catheter is markedly reduced. That is to say the windings of the wire helix, which form a rib-like surface, in this case result in high friction acting in the longitudinal direction of the catheter between the wire helix and the tubular hollow body.

Additionally, it was found to be advantageous for the wire helix to extend from a front end of the expandable tubular hollow body to a rear end of the expandable tubular hollow body in a longitudinal direction which runs parallel to the longitudinal axis of the catheter. By way of example, if the wire helix is provided as support for a radiopaque marking, any position of the expandable tubular hollow body, such as, in particular, the front end and/or the rear end therefore in principle can be marked. This arrangement also has advantages in respect of slippage of a tubular hollow body borne directly on the wire helix because the rib-like surface of the wire helix namely is present over the entire length of the expandable tubular hollow body.

However, it is also possible for the wire helix to have a shorter design such that a wire helix is merely found in a portion of the region encased by the expandable tubular hollow body. By way of example, this can be in the region of the front end, but also in the region of the rear end, of the expandable tubular hollow body.

It was additionally found to be advantageous for the wire helix to be arranged within a hollow shaft of the catheter arranged within the encased region, wherein it is preferably an innermost hollow shaft of the catheter. Here, a longitudinal axis of the hollow shaft is arranged coaxially, in particular, with respect to the longitudinal axis of the wire helix. This ensures that the wire helix does not lead to an increase in the catheter diameter as an externally arranged device. Moreover, any sharp-edged protrusions of the wire helix are covered as best as possible by the hollow shaft and so mechanically sensitive, expandable tubular hollow bodies, such as balloons, can also be attached to the catheter.

However, in principle, the wire helix can also be arranged outside of a hollow shaft should this be expedient for other reasons. Additionally, the wire helix can also be arranged only partly within a hollow shaft, for example with the rear end of said wire helix. In this case, the front end of the wire helix can protrude out of the hollow shaft and, for example, it can be directly surrounded by the expandable tubular hollow body. However, it is also within the scope of the invention for the rear end of the wire helix to be arranged within an external hollow shaft while the front end of the hollow shaft is borne on the outside of an internal shaft of the catheter.

If the wire helix is arranged within a hollow shaft, the hollow shaft in the process is particularly preferably fitted to the wire helix such that an outer surface of the hollow shaft substantially corresponds to an outer contour of the wire helix. Hence, the region of the outer surface of the hollow shaft also has a rib-like surface that particularly reduces the risk of the expandable tubular hollow body slipping, as already explained above. However, it is also possible to dispense with the fit. In this case, the frictional force between the expandable tubular hollow body and the hollow shaft reduces correspondingly. In principle, it is also possible for, for example, an embossed structure or protruding ribs, in particular running transversely with respect to a longitudinal direction of the hollow shaft, to be attached to the outer surface of the hollow shaft in addition to the fit or instead of the fit.

During the production of a catheter, the wire helix is preferably inserted into a hollow shaft of the catheter prior to an application of the expandable tubular hollow body and the hollow shaft is subsequently fitted to an outer contour of the wire helix. By way of example, this can be brought about by heating the hollow shaft. However, it can also be sufficient for a relatively closely fitting wire helix to be inserted into the hollow shaft. Since the hollow shafts conventionally used in catheters are usually made of soft and flexible material, and moreover are thin-walled, such hollow shafts can also be matched to the contour of the wire helix on their own accord.

The wire helix advantageously has an external diameter of 0.2-0.5 mm and a wire diameter of 30-100 μm. It was found that such wire helices have the required flexibility but at the same time still have sufficient stiffness. The external diameter of 0.2-0.5 mm moreover corresponds to the internal diameters of catheter shafts conventionally used these days. The wire helix could therefore be integrated in catheter shafts already available commercially, which helps in keeping down the production costs of the catheters according to the invention. However, in principle, differently dimensioned wire helices could also be used. However, if, for example, the wire diameter thereof is too large, the flexibility of the catheter is reduced.

In particular, a straight-line inner wire can be attached within the wire helix, with the former being connected to the wire helix in a punctiform fashion at one or more connection regions. By way of example, this can modify the flexibility and/or the stiffness of a wire helix. Wire helices with a small external diameter, which have high flexibility, thus can be made stiffer, for example. The connection between the wire helix and the inner wire can for example be brought about by a cohesive connection technique, in particular by brazing or welding.

The wire helix and the straight-line inner wire are preferably brazed together in the connection regions using radiopaque filler, more particularly silver filler. This can form radiopaque regions of the catheter in a simple fashion. Radiopaque regions are particularly important for checking the precise position of the catheter in the hollow organ. In this context and in the following text, “radiopaque regions” are understood to mean that the radiopaque regions can be distinguished from the surrounding regions of the catheter and/or from the surrounding tissue due to the absorption of impinging X-ray radiation in an imaging method of the human and/or animal body, wherein the irradiated X-ray radiation is of a usual dose that is safe for the human and/or animal body. Thus, the otherwise conventionally attached, bulky radiopaque rings arranged around individual shafts of the catheter can be dispensed with, and this helps in keeping the dimensions of the catheter small. The advantageously utilized silver filler is additionally harmless for the human and/or animal body and so there is no danger to the patient or the animal from this material.

It is particularly advantageous for the wire helix itself to consist of radiopaque material, more particularly platinum, gold or tungsten. Here, radiopaque materials are distinguished by large absorption cross sections for X-ray radiation. Stainless steel, which has a much smaller absorption cross section compared to platinum, gold or tungsten, cannot be imaged in X-ray imaging of the human and/or animal body under the conventional conditions and is therefore not radiopaque but radiolucent. If the wire helix itself is produced from radiopaque material, radiopaque rings can likewise be dispensed with for forming radiopaque regions on the catheter in the case of a sufficiently long wire helix. Since this means that in principle no more additional radiopaque material at all has to be arranged on the catheter, this allows a particularly simple catheter design.

However, it is also possible, for example, to arrange a wire helix made of radiopaque material in a front region of the expandable tubular hollow body and to attach a radiopaque marking ring and/or radiopaque filler in a rear region of said body. This already significantly increases the ability to insert the catheter because at least the front region of the catheter can have a smaller diameter.

A plurality of adjacent windings of the wire helix are preferably arranged abutting one another and thus form a radiopaque region of the catheter. This is because it was found that at least five adjacent windings of the wire helix made of radiopaque material should abut one another to form a radiopaque region in order to obtain an optimum contrast in the imaging method using X-rays in the human and/or animal body. It is also possible that the windings are not arranged directly abutting one another or that provision is made of less than five abutting windings as a radiopaque region. However, the contrast in the imaging method reduces accordingly.

It was found that regions in the wire helix, in which there is a spacing between the individual windings corresponding to at least double, more particularly triple, the wire diameter, are no longer visible in the imaging method using X-ray radiation in the human and/or animal body. Here, the actual free space between the windings is decisive as the spacing. In this context, a winding is understood to be a region of the wire helix running precisely once around the longitudinal axis of the wire helix.

Therefore, adjacent windings are preferably arranged without touching and spaced apart for producing a radiolucent region of the wire helix made of radiopaque material, and so, in particular, there is a spacing between the individual windings corresponding to at least double, preferably triple, the wire diameter.

This can mark individual regions of the catheter as radiopaque in a targeted fashion, while other regions have a radiolucent design. However, it is also possible to use a wire helix made of radiopaque material that comprises abutting windings over its entire length and thus is completely radiopaque.

In particular, a plurality of radiopaque regions and a plurality of radiolucent regions of the wire helix can be arranged alternately and more particularly at regular intervals for marking the length. The visible length markings in the X-ray image for example supply the medical practitioner with information relating to the position of the catheter relative to the image plane of the X-ray image. By way of example, if the length markings are situated closely together, the catheter extends out of the image plane in one direction. If the length markings show the maximum spacing, the catheter is situated parallel to the image plane. If the position of the catheter is imaged from different directions, it is therefore in principle possible to determine the spatial position of the catheter. The length markings do not necessarily have to be present in the encased region of the catheter. It is also possible to arrange them in a region of the wire helix that extends backward from the encased region.

During the production of a catheter with such a wire helix, the latter is compressed in a first region and stretched in a second region, which directly adjoins the first region, prior to an application of the expandable tubular hollow body. This is repeated as often as necessary, until a desired sequence of radiopaque and radiolucent regions is present.

The solution according to the invention is particularly suitable if there are at least two expandable tubular hollow bodies arranged lying coaxially above one another. Such catheters usually have a relatively large external diameter in the region of the two expandable tubular hollow bodies. Even small reductions in the external diameter in this case help markedly to improve the ability to insert the catheter because the cross-sectional area increases with the square of the diameter or radius. The wire helix according to the invention thus affords keeping the external diameter or the cross-sectional area of the catheter as small as possible. However, good stability and flexibility are ensured at the same time, as result of which it is easier to insert the catheter. However, it is also possible for merely a single expandable tubular hollow body to be provided.

In particular, an actuatable balloon is arranged as expandable tubular hollow body. In principle, the balloon can be arranged around the wire helix in a folded-up fashion. In this case, the wire helix allows particularly effective actuation of the balloon. This is because a fluid can be guided directly into the folded-up balloon through the free spaces between the individual windings of the wire helix. The actuation is then brought about along the entire length of the wire helix allowing even and rapid actuation of the balloon. However, it is also possible to arrange the balloon around a wire helix arranged in a shaft. By way of example, the actuation can then be brought about through special openings in the shell of the hollow shaft. The wire helix arranged in the interior of the hollow shaft then does not constitute a particularly big resistance to the fluid guided in the hollow shaft, and so the balloon also can be actuated in this case. If the hollow shaft is additionally fitted to a contour of the wire helix, the fluid entering between the hollow shaft and the folded-up balloon during the actuation can be better distributed along the hollow shaft in particular. This is because the fluid can spread in an improved fashion due to the uneven surface of the hollow shaft, leading to a more even actuation of the balloon. A more even actuation is understood to mean that the balloon is evenly widened along its entire length. This is particularly important if the balloon is in the region of a stenosis in a hollow organ for example that should be widened. If, in the process, part of the balloon is behind or in front of the stenosis, the position of the catheter in the hollow organ can be displaced and it can slip out of the region of the stenosis in the case of uneven actuation. This risk is much smaller in the case of even actuation because the part of the balloon in the stenosis is also actuated from the outset and hence the balloon is trapped in the stenosis.

As described above, it is now possible to realize a radiopaque region directly in the wire helix. For example, radiopaque silver filler can be introduced into the wire helix or the wire helix is directly manufactured from radiopaque material. However, in both cases the radiopaque region does not lead to an increase in the external diameter of the catheter.

In particular, the balloon can have a coating, wherein the coating contains at least one medicament. The coating can then be pushed against the walls of the hollow organ in a region to be treated and so the medicament can be taken up by the hollow organ. Since such coatings likewise have a significant layer thickness, it is also very advantageous in this case for the catheter with a tightly fitting balloon to be designed with the smallest possible diameter.

By way of example, a medical implant, more particularly a stent, can also be arranged as an expandable tubular hollow body, which is preferably fitted to an outer contour of the wire helix. If the medical implant is situated directly on the wire helix and/or a hollow shaft of the catheter fitted to the contour of the wire helix as the only expandable tubular hollow body, this also has great advantages. This is because the uneven structure of the wire helix and/or the fitted hollow shaft ensures that the medical implant is not displaced forward or backward along the axial direction or even stripped off the catheter during the insertion of the catheter into the hollow organ.

A medical implant can also have a coating, wherein the coating contains at least one medicament. As in the case of the catheter with balloon, the wire helix according to the invention allows a very compact design of the catheter, even in the case of a medical implant, which greatly improves the ability to insert the catheter.

The catheter tip is preferably designed as a flexible helical spring. In the process, it is possible for a continuous wire helix to be arranged extending from the tip of the catheter into the encased region and, as the case may be, out of the latter again at the rear end. This simplifies the production of the catheter according to the invention in particular. However, the provision of a tip with a different shape, which tip e.g. has been optimized in respect of a particular application, is also possible.

In the case of a catheter to be inserted into a hollow organ of a human or an animal, comprising a forwardly projecting catheter tip and a hollow shaft, it is also possible for a radiopaque ring, more particularly made of platinum, gold or tungsten, to be arranged on the hollow shaft and for a plurality of rib-like projections surrounding the hollow shaft and projecting from the latter to be attached to the hollow shaft in a region in front of and/or behind the radiopaque ring. The ability to insert such a catheter is likewise improved. In order to produce the latter, a radiopaque ring is preferably attached to the hollow shaft in a first method step. The hollow shaft is subsequently compressed in a longitudinal direction of the hollow shaft in a plurality of regions in front of the radiopaque ring and/or in a plurality of regions behind the radiopaque ring for the formation of rib-like projections.

It was found that such rib-like projections particularly improve the elasticity and flexibility of the hollow shaft. Additionally, the rib-like projections projecting from the hollow shaft act as edge protection for the radiopaque ring. This is very advantageous, particularly in the case of catheters with balloons, because the balloon sheaths that are usually very sensitive to mechanical influences are protected against damage in the best possible fashion. Moreover, the radiopaque ring is at the same time fixed in the axial direction. It is thus practically impossible to displace the ring when inserting the catheter into a hollow organ.

Further advantageous embodiments and feature combinations of the invention emerge from the following detailed description and the entirety of the patent claims.

### BRIEF DESCRIPTION OF THE DRAWINGS

In the drawings used to explain the exemplary embodiment:

FIG. 1 shows a cross section through a catheter with a wire helix in a hollow shaft and, arranged around it, a folded-up balloon that is surrounded by a stent;

FIG. 2 shows a cross section through a catheter with a wire helix that is directly surrounded by a stent; and

FIG. 3 shows a cross section through a catheter with a wire helix in a front region of a hollow shaft and a radiopaque marking ring on the hollow shaft, wherein the hollow shaft has rib-like projections in front of and behind the marking ring.

In the figures, the same parts in principle have been denoted by the same reference sign.

### WAYS OF IMPLEMENTING THE INVENTION

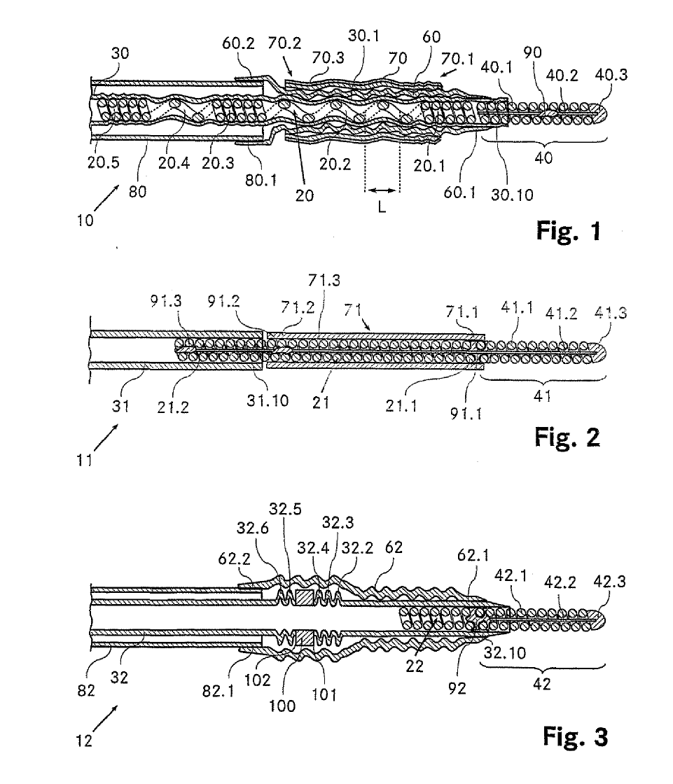

FIG. 1 illustrates a cross section of a first catheter 10 according to the invention. The catheter 10 comprises an outer shaft 80, from which a hollow shaft 30 projects to the right in the longitudinal direction. The rear end of a catheter tip 40 is anchored in the hollow shaft 30 at the front end 30.10 of the hollow shaft 30 and so the catheter tip 40 protrudes out of the hollow shaft 30 in the longitudinal direction. The catheter tip 40 consists of a cylindrical helical spring 40.1, which has a support wire 40.2 in the center, the support wire running along the entire length of the helical spring 40.1 for stabilization purposes. Here, the support wire 40.2 is connected to the helical spring 40.1 in approximately the center of the longitudinal direction of the helical spring 40.1 in a cohesive fashion by a braze point 90 made of silver filler. Additionally, at the front end of the helical spring 40, there is a rounded end cap 40.3. For the purposes of anchoring the helical spring 40, the rearmost windings of the helical spring 40.1 arranged in the interior of the hollow shaft 30 are pressed into the front end 30.10 of the hollow shaft 30.

Additionally, a wire helix 20 made of platinum is arranged coaxially in the hollow shaft 30 in a region behind the helical spring 40.1. The wire helix has e.g. an external diameter of 0.4 mm, wherein the wire thickness of the wire helix 20 is 50 μm, for example. In a frontmost or first region 20.1, the wire helix 20 has five abutting windings. The first region 20.1 of the helical spring 20 is radiopaque due to the material (platinum) and the density of the windings. In a second region 20.2 adjoining the first region 20.1, the wire helix 20 has three windings attached completely without contact, wherein there is a spacing L in the longitudinal direction of approximately 160 μm between two windings. The spacing L corresponds to approximately three times the wire thickness of the wire helix 20. Due to the low density of the windings, the second region 20.2 of the helical spring 20 is radiolucent. The second region 20.2 of the wire helix 20 is adjoined by the third region 20.3, which again comprises five abutting windings of the helical spring and is therefore likewise radiopaque. The fourth region 20.4 of the helical spring 20 situated behind the third region 20.3 comprises a winding arranged without contact, as result of which this region is radiolucent. The fourth region 20.4 is adjoined by the fifth region 20.5 of the helical spring, which fifth region has substantially the same design as the third region 20.3 of the helical spring 20. Further regions of the helical spring 20 not illustrated in FIG. 1 are arranged behind the fifth region 20.5. In the process, regions with windings arranged without contact (analogous to the fourth region 20.4) alternate with regions with abutting windings (analogous to the third region 20.3) and form a regular pattern of radiopaque and radiolucent regions.

The hollow shaft 30 is fitted throughout to the outer shape or contour of the wire helix 20. The outer surface 30.1 of the hollow shaft 30 therefore has a screw-like structure, wherein a region with short pitch and a region with long pitch alternate.

The rear end 60.2 of an actuatable and expandable balloon 60 is additionally attached to the front end 80.1 of the outer shaft 80 over the entire circumference of the outer shaft 80. Here, the balloon 60 is designed as a tubular hollow body and arranged such that its longitudinal axis is coaxial to the longitudinal axis of the catheter 10. Here, the part of the balloon 60 situated in front of the outer shaft 80 is situated directly on the hollow shaft 30 with the screw-like structure and the front end 60.1 of said part of the balloon 60 is welded to the hollow shaft 30 in the region of the front end 30.10 thereof. Here, the first two regions 20.1, 20.2 of the wire helix are completely surrounded by the balloon 60.

Moreover, a stent 70 or a tubular medical implant that can be expanded by the balloon 60 is attached outside of the balloon 60 and likewise coaxially to the longitudinal axis of the catheter. Here, the stent 70 is pressed against the balloon 60 from the outside and fitted to the outer surface 30.1 of the hollow shaft 30 or to the contour of the wire helix 20 in the first two regions 20.1, 20.2. Here, the rear end 70.2 of the stent 70 is situated in front of the rear end 60.2 of the balloon 60. At the same time, the front end 70.1 of the stent 70 is situated behind the front end 60.1 of the balloon 60.

Additionally, the stent 70 is surrounded in the region of the outer shell surface by a coating 70.3 containing a medicament.

When the balloon 60 is actuated, the fluid used in the process can expand over the entire length of the balloon 60 due to the screw-shaped structure of the outer surface 30.1 of the hollow shaft 30, as a result of which there is an even actuation of the balloon 60.

This also reduces the risk of the stent 70 being able to be displaced or even slipping off the catheter 10 in the longitudinal direction thereof during the actuation of the balloon 60.

FIG. 2 shows the cross section of a second catheter 11 according to the invention. The catheter 11 has a hollow shaft 31, in which a wire helix 21 made of stainless steel, which protrudes out of the hollow shaft 31, is arranged coaxially in a region of the front end 31.10. The wire helix 21 has an external diameter of for example 0.5 mm and a wire diameter of e.g. 100 μm. A straight-line support wire 41.2 is attached in the interior of the wire helix 21. In the region of the front end 21.1 of the wire helix 21, said wire helix is brazed to a catheter tip 41 with a first braze point 91.1 of silver filler. The catheter tip 41 consists of a helical spring 41.1 made of stainless steel and it has a rounded end cap 41.3 at its front end.

A support wire 41.2 is arranged in the interior of the helical spring 41.1, which extends from the end cap 41.3 of the catheter tip 41 and through the first braze point 91.1 and the wire helix to the rear end 21.2 of the latter. Just in front of the front end 31.10 of the hollow shaft 31, there is a second braze point made of sliver filler, which interconnects the support wire 41.2 and the wire helix. At the rear end of the wire helix, there is a third braze point which connects the rear end of the support wire 41.2 to the wire helix 21.

Directly in front of the front end 31.10 of the hollow shaft 31, a cylindrical stent 71 or a tubular medical implant is arranged around the wire helix 21. The rear end 71.2 of the stent 71 surrounds the second braze point 91.2, while the first braze point 91.1 is surrounded by the front end 71.1 of the stent 71. There are sufficient amounts of silver filler at the three braze points 91.1, 91.2, 91.3 in this case and so these are radiopaque.

The stent 71 is additionally pressed against the wire helix 21, and so irregularities (not illustrated in FIG. 2) of the inner surface 71.3 of the tubular stent 71 are pressed into the intermediate spaces between the individual windings of the wire helix 21. This significantly hinders a displacement of the stent in a longitudinal direction of the catheter 11, which is particularly advantageous during the insertion of the catheter 11 because the stent 71 can hardly slip off the catheter 11 as a result of this.

The stent 71 is designed as a self-expandable hollow body and is kept in the compressed form illustrated in FIG. 2 by an envelope (not illustrated).

A third catheter 12 according to the invention is illustrated in FIG. 3. Here, a hollow shaft 32 extends in an outer shaft 82, and the former protrudes toward the front out of the front end 82.1 of the outer shaft 82. A radiopaque ring 100, for example made of platinum, is arranged around the hollow shaft 32 in a region in front of the front end 82.1 of the outer shaft 82. The hollow shaft 32 has three ribs 32.2, 32.3, 32.4, completely encircling the hollow shaft 32, arranged behind one another in a region directly in front of the radiopaque ring 100. Here, the three ribs 32.2, 32.3, 32.4 protrude outward from a shell surface of the hollow shaft 32 and consist of compressed material of the hollow shaft 32 made of plastic. Two further ribs 32.5, 32.6 are arranged behind the radiopaque ring 100 in the same fashion. Here, the five ribs 32.2 . . . 32.6 of the hollow shaft 32 have an external diameter corresponding to the external diameter of the radiopaque ring 100. As a result, the radiopaque ring 100 is embedded between the three front ribs 32.2, 32.3, 32.4 and the two rear ribs 32.5, 32.6. In particular, the rear edge 102 and the front edge 101 of the radiopaque ring 100 are thus covered in the longitudinal direction of the catheter 11 by the ribs 32.2 . . . 32.6.

In the region of the front end 32.10 of the hollow shaft 32, a helical spring 42.1 made of stainless steel is anchored in the hollow shaft 32 as component of a catheter tip 42. The helical spring 42.1 has a support wire 42.2 extending in the longitudinal direction in the interior and a rounded end cap 42.3 at the front end.

A wire helix 22 with six windings is arranged in the hollow shaft 32 behind the helical spring 42.1, the helix being connected to the rear end of the helical spring 42.1 by a braze point 92. The wire helix consists of tungsten and has an external diameter of for example 0.2 mm. The wire thickness of the wire helix is e.g. 30 μm. Due to the windings of the wire helix 22 abutting one another, the latter is radiopaque.

Furthermore, outside of the hollow shaft 32, an actuatable balloon 62 is arranged around the hollow shaft 32. Here, the front end 62.1 of the balloon 62 is welded to the hollow shaft 32 in the region of the front end 32.10 thereof. The rear end 62.2 of the balloon 62 is welded to the outside of the outer shaft 82 at the front end 82.1 thereof.

During the production of the catheter 12 from FIG. 3, a radiopaque ring 100 is pushed onto a hollow shaft 32 in a first step. Subsequently, the hollow shaft 32 is compressed, for example in the region in front of the radiopaque ring 100, to form a first rib 32.4 and it is subsequently stretched slightly again. A second rib 32.3 and a third rib 32.2 are formed in an analogous fashion. Subsequently, the ribs 32.5, 32.6 are produced in the same way behind the radiopaque ring 100.

The described exemplary embodiments should be understood as illustrative examples, which can be extended or modified arbitrarily within the scope of the invention.

Thus, for example, instead of the three described catheter tips 40, 41, 42, it is also possible to provide catheter tips that have a different suitable flexible device instead of the helical springs 40.1, 41.1, 42.1. It is likewise possible for the helical springs 40.1, 41.1, 42.1 of the catheter tips to be produced from a radiopaque material, such as gold, platinum or tungsten, and so the entire catheter tip is radiopaque. A further option consists of forming the three wire helices 20, 21, 22 and the three helical springs 40.1, 41.1, 42.1 from a single helical spring, made in particular from a radiopaque material, which according to the invention extends as far as the regions of the catheter encased by the expandable hollow bodies.

In the case of the first catheter 10 described in FIG. 1, the number of windings of the wire helix 20 abutting one another and/or number of windings arranged without contact can be increased, for example to obtain better visibility during X-raying. For this, it is also possible to attach additional radiopaque silver filler in the radiopaque regions of the wire helix 20.

Moreover, the wire helix 20 in the first catheter 10 does not necessarily have to protrude into the region of the outer shaft 80. It is also possible that provision is made for a wire helix which, as in the case of the third catheter 12 in FIG. 3, merely protrudes into a front region of the balloon 60 or stent 70.

It is also possible for the coating 70.3 of the stent 70 to be omitted in the case of the first catheter 10, or to be replaced by a different coating. By way of example, coatings of silicone which improve the sliding properties of the catheter are particularly suitable.

It is also possible for the stent 70 to be omitted in the first catheter 10 and so the balloon 60 is present as the outermost expandable hollow body. In this case, e.g. the balloon can also be provided with a coating with medicaments or a coating for improving the gliding properties.

In the case of the second catheter 11 from FIG. 2, the wire helix 21 can also be stretched or designed such that the individual windings of the wire helix are arranged without contact in the region of the stent 71. This possibly affords a further increase in the friction between the stent 71 and the wire helix 21. In the case of the second catheter, rather than using a wire helix made of stainless steel, it is also possible to use a wire helix made of a radiopaque material such as gold, platinum or tungsten.

In addition to the radiopaque ring 100 shown in FIG. 3, it is also possible for further radiopaque rings to be arranged on a shaft of the catheter.

In summary, it should be noted that a catheter with an expandable casing and a novel design is provided, which in particular has a very compact design. This is accompanied with significant advantages when inserting the catheter because the latter can be guided through stenoses in a hollow organ or a vessel in the best possible fashion due to the small external diameter. Additionally, the catheter according to the invention can be produced economically.

1. A catheter to be inserted into a hollow organ of a human or an animal, comprising a forwardly projecting catheter tip, wherein a region encased by an expandable tubular hollow body is arranged behind the catheter tip, characterized in that a wire helix is arranged in the encased region.

2. The catheter as claimed in claim 1, characterized in that a longitudinal axis of the wire helix is arranged coaxially to a longitudinal axis of the catheter and in that, more particularly, the wire helix extends from a front end of the expandable tubular hollow body to a rear end of the expandable tubular hollow body in a longitudinal direction, which runs parallel to the longitudinal axis of the catheter.

3. The catheter as claimed in claim 1, characterized in that the wire helix is arranged within a hollow shaft of the catheter arranged within the encased region, wherein it is preferably an innermost hollow shaft of the catheter.

4. The catheter as claimed in claim 1, characterized in that the hollow shaft is fitted to the wire helix such that an outer surface of the hollow shaft substantially corresponds to an outer contour of the wire helix.

5. The catheter as claimed in claim 1 characterized in that the wire helix has an external diameter of 0.2-0.5 mm and a wire diameter of 30-100 μm.

6. The catheter as claimed in claim 1, characterized in that a straight-line inner wire is attached within the wire helix, with the former being connected to the wire helix in a punctiform fashion at one or more connection regions.

7. The catheter as claimed in claim 6, characterized in that the wire helix and the straight-line inner wire are brazed together in the connection regions using radiopaque filler, more particularly silver filler, wherein the connection regions form radiopaque regions of the catheter.

8. The catheter as claimed in claim 1, characterized in that the wire helix consists of radiopaque material, more particularly platinum, gold or tungsten.

9. The catheter as claimed in claim 8, characterized in that a plurality of adjacent windings of the wire helix are arranged abutting one another and thus form a radiopaque region of the catheter.

10. The catheter as claimed in claim 7, characterized in that adjacent windings are arranged without touching and spaced apart for producing a radiolucent region of the wire helix, and so there preferably is a spacing between the individual windings corresponding to at least double, more particularly triple, the wire diameter.

11. The catheter as claimed in claim 10, characterized in that a plurality of radiopaque regions and a plurality of radiolucent regions of the wire helix are arranged alternately and more particularly at regular intervals for marking the length.

12. The catheter as claimed in claim 1, characterized in that there are at least two expandable tubular hollow bodies arranged lying coaxially above one another.

13. The catheter as claimed in claim 1, characterized in that an actuatable balloon is arranged as expandable tubular hollow body.

14. The catheter as claimed in claim 13, characterized in that the balloon has a coating, wherein the coating contains at least one medicament.

15. The catheter as claimed in claim 1, characterized in that a medical implant, more particularly a stent, is arranged as an expandable tubular hollow body, which is preferably fitted to an outer contour of the wire helix.

16. The catheter as claimed in claim 15, characterized in that the medical implant has a coating, wherein the coating contains at least one medicament.

17. The catheter as claimed in claim 1, characterized in that the catheter tip is basically designed as a flexible helical spring.

18. A catheter, more particularly as claimed in claim 4, to be inserted into a hollow organ of a human or an animal, comprising a forwardly projecting catheter tip and a hollow shaft, characterized in that a radiopaque ring, more particularly made of platinum, gold or tungsten, is arranged on the hollow shaft, wherein the hollow shaft has a plurality of rib-like projections surrounding the hollow shaft and projecting from the latter in a region in front of and/or behind the radiopaque ring.

19. A method for producing a catheter, more particularly a catheter as claimed in claim 1, characterized in that a wire helix is surrounded by an expandable, tubular hollow body, more particularly a stent and/or an actuatable balloon.

20. The method as claimed in claim 19, characterized in that the wire helix is compressed in a first region and stretched in a second region, which directly adjoins the first region, prior to an application of the expandable tubular hollow body.

21. The method as claimed in claim 19, characterized in that the wire helix is inserted into a hollow shaft of the catheter prior to an application of the expandable tubular hollow body and the hollow shaft is subsequently fitted to an outer contour of the wire helix.

22. A method for producing a catheter, more particularly a catheter as claimed in claim 18, characterized in that a radiopaque ring is attached to the hollow shaft and in that the hollow shaft is subsequently compressed in a longitudinal direction of the hollow shaft in a plurality of regions in front of the radiopaque ring and/or in a plurality of regions behind the radiopaque ring for the formation of rib-like projections.
